(19) United States
(12) Patent Application Publication
(10) Pub. No.: US 2011/0263870 A1
(43) Pub. Date: Oct. 27, 2011

(54) NOVEL PYRROLE DERIVATIVES AND THEIR SYNTHESIS

(75) Inventor: Braja SUNDAR PRADHAN, Hyderabad (IN)

(73) Assignee: HELVETICA INDUSTRIES (P) LIMITED, New Delhi (IN)

(21) Appl. No.: 13/091,717

(22) Filed: Apr. 21, 2011

(30) Foreign Application Priority Data
Apr. 23, 2010 (IN) 2197/DEL/2009

(51) Int. Cl. C07F 9/572; C07D 207/34

(52) U.S. Cl. 548/413; 548/531

(57) ABSTRACT

The present invention relates to two novel pyrrole derivatives [3-Phenyl-4-(phenylcarbamoyl)-2-(4-fluorophenyl)-5-(1-methylethyl)-pyrrole-1-yl]methyl(diphenyl)phosphine oxide and Diethyl [3-Phenyl-4-(phenylcarbamoyl)-2-(4-fluorophenyl)-5-(1-methylethyl)-pyrrole-1-yl]methylphosphonate. These pyrrole derivatives can be used as intermediates for the synthesis of the anticholesterol drug atorvastatin.

### FIELD OF INVENTION

The present invention relates to two novel pyrrole derivatives of formula I and formula II i.e. [3-Phenyl-4-(phenylcarbamoyl)-2-(4-fluorophenyl)-5-(1-methylethyl)-pyrrole-1-yl]methyl(diphenyl)phosphine oxide I and Diethyl [3-Phenyl-4-(phenylcarbamoyl)-2-(4-fluorophenyl)-5-(1-methylethyl)-pyrrole-1-yl]methylphosphonate II. These pyrrole derivatives are intermediates for the synthesis of atorvastatin. The invention also relates to processes for synthesis of the above novel pyrrole derivatives. The pyrrole derivatives have the following structures:

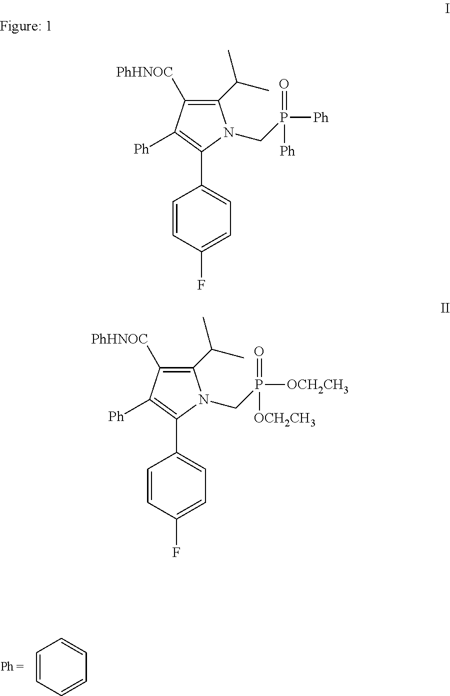

### BACKGROUND OF THE INVENTION

Atorvastatin is a synthetic lipid lowering agent that acts as an inhibitor of 3-hydroxy-3-methylglutaryl-coenzyme A (HMG-CoA). HMG-CoA is a key enzyme in the biosynthesis of cholesterol in humans. Its competative inhibition leads to a reduction in the rate of biosynthesis of cholesterol. Atorvastatin is indicated for use for reducing elevated total cholesterol, low density lipoprotein cholesterol, apolipoprotein B and high plasma triglycerides in patients with primary hypercholesterolemia and mixed hyperlipidemia.

In the known art, the synthesis of atorvastatin has been described in the literature, for example, U.S. Pat. No. 5,155,251, U.S. Pat. No. 5,103,024, EP-B 330172, and it is achieved by condensing the amine component with the atorvastain 1,4-diketone component, 2-[1-Phenyl-2-(4 fluorophenyl)-2-oxo-ethyl]-4-methyl-N-methyl-N-phenyl-3-oxo-pentamide, under the catalysis of pivalic acid at reflux temperature of the solvent system used in the reaction (FIG. 2).

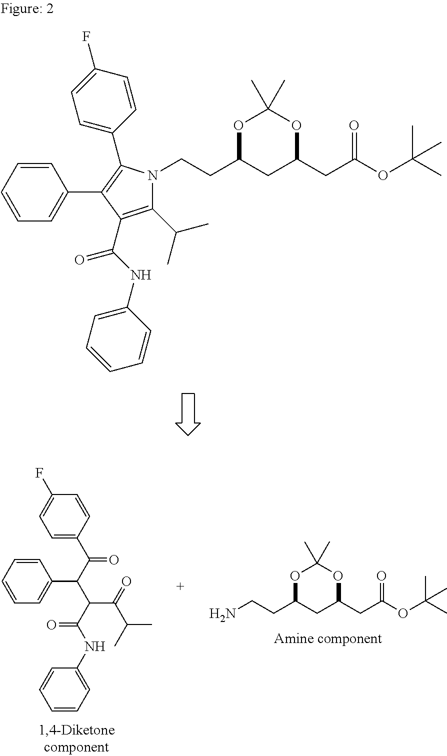

In condensation such as this, the components are often not completely consumed during the reaction process necessitating the removal of these unreacted components, in particular the atorvastatin 1,4-diketone component, by a laborius process of purification to obtain the desired pyrrole derivative in a fairly pure form. In order to address this problem, the pyrrole derivatives I and II have been devised (FIG. 1).

### SUMMARY OF THE INVENTION

According to an object of invention there are provided the pyrrole derivatives I or II which are new chemical entities.

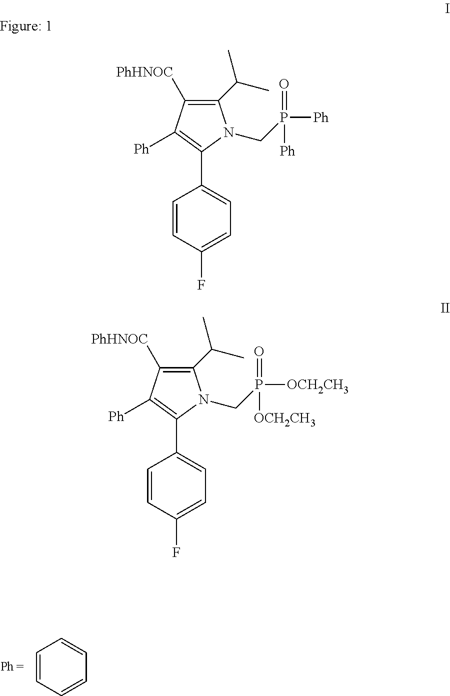

The present invention also relates to synthesis of new pyrrole derivatives I [3-Phenyl-4-(phenylcarbamoyl)-2-(4-fluorophenyl)-5-(1-methylethyl)-pyrrole-1-yl]methyl(diphenyl)phosphine oxide, the compound of formula I and Diethyl [3-Phenyl-4-(phenylcarbamoyl)-2-(4-fluorophenyl)-5-(1-methylethyl)-pyrrole-1-yl]methylphosphonate from N-(bromomethyl)phathalimide.

For synthesis of pyrrole derivative I, N-(bromomethyl)phathalimide III reacts with ethyl diphenylphosphinite (Scheme: 1) to give diphenyl(phthalimidomethyl)phosphine oxide IV which, on treatment with aqueous hydrobromic acid gets converted into aminomethyl (diphenyl)phosphine oxide V. The phosphine oxide then condenses with the atorvastatin 1,4-dicarbonyl compound VI to give the pyrrole derivative I (Scheme: 2).

According to another aspect of the invention there is provided a process for synthesis of pyrrole derivative II by reacting N-(bromomethyl)phthalimide III with triethyl phosphite under “Arbuzov” reaction conditions to furnish diethyl phthalimidomethylphosphonate VII, which reacted with hydrazine hydrate (Scheme: 3) to furnish diethyl aminomethylphosphonate VIII. The phosponate then condenses with the atorvastatin 1,4-dicarbonyl compound VI to give the pyrrole derivative II.

According to another aspect of the invention there is provided a process for conversion of novel pyrrole derivatives I and II into a furanose moiety.

According to another aspect of the invention the pyrrole derivative I under suitable Wittig reaction condition should be converted into the furanose moiety X by reacting it with a known aldehyde IX moiety (Scheme: 4). The hydroxy comound X, on treatment with a suitable base, converted into furanose compound XI.

According to another aspect of the invention the pyrrole derivative II under suitable “Wittig” reaction conditions should be converted into the furanose moiety XI in a single step (Scheme: 5) without the formation of the intermediate hydroxyl compound X.

According to another invention the furanose moiety XI should, on reduction, furnish the furanose derivative XII which may be converted into atorvastatin by known method as described in prior arts.

### DETAILED DESCRIPTION OF THE INVENTION

For the preparation of the pyrrole derivatives I and II, N-(bromomethyl) phthalimide III serves as the starting material.

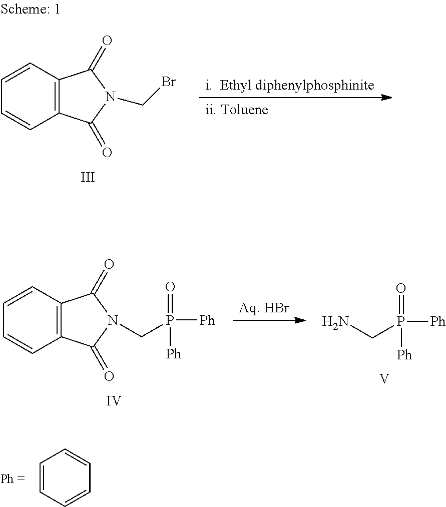

The phthalimide compound III reacts with ethyl diphenylphosphinite under “Arbuzov” reaction conditions to give diphenyl (phthalimidomethyl)phosphine oxide IV which, on treatment with aqueous hydrobromic acid, converted to aminomethyl(diphenyl)phosphine oxide V (Scheme: 1). The phosphine oxide V then condenses with the 1,4-dicarbonyl compound VI to give the pyrrole derivative I (Scheme: 2).

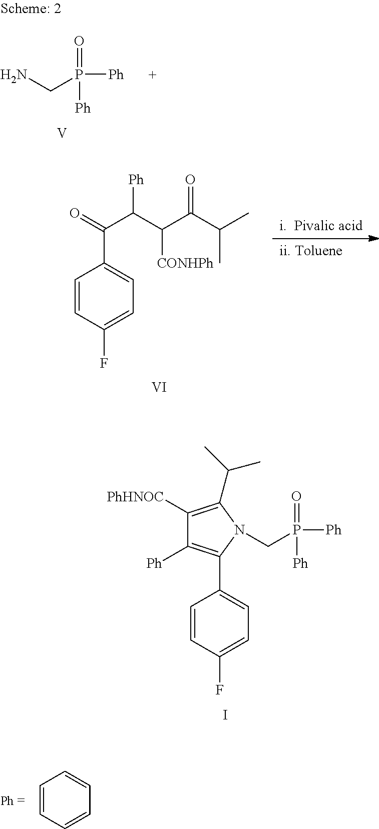

In a similar fashion, N-(bromomethyl)phathalimide III reacts with triethyl phosphite under “Arbuzov” reaction conditions to furnish diethyl phthalimidomethylphosphonate VII which reacted with hydrazine hydrate to furnish diethyl aminomethylphosphonate VIII (scheme 3).

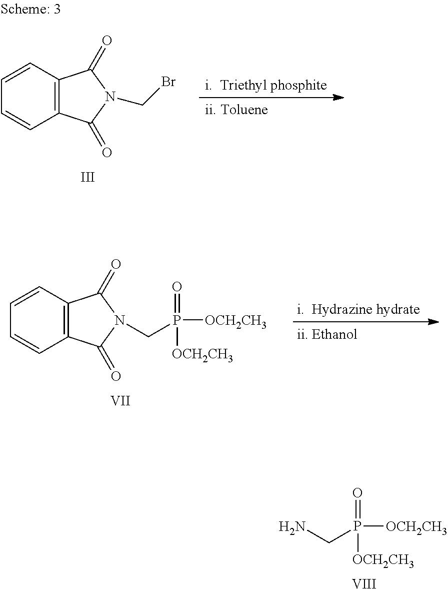

The phosponate VIII reacts with the 1,4-dicarbonyl compound VI to furnish the pyrrole derivative II.

### Example 1

### Diphenyl(phthalimidomethyl)phosphine oxide IV

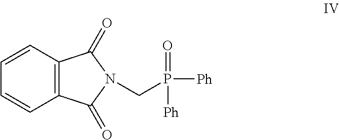

Toluene (25 ml) was added to a mixture of ethyl diphenylphosphinite (5 g, 21.71 mmole) and the N-(bromomethy)phthalimide III (5.21 g, 21.70 mmole) at room temperature. The reaction mixture was heated to 90° C. and maintained at this temperature for 48 h when the reaction was complete as indicated from the TLC of the reaction mixture.

The reaction mixture was concentrated under reduced pressure, triturated with toluene and filtered to furnish the phosphine oxide compound IV as a solid; yield: 6 g, 76.5%.

### Example 2

### Aminomethyl(diphenyl)phosphine oxide V

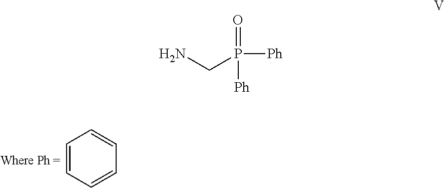

Aqueous hydrobromic acid (49% w/w; 40 ml) was added to the phosphine oxide compound IV (10 g, 27.70 mmole) at room temperature and the reaction mixture was heated to 100° C. After 15 h at this temperature, the reaction mixture was allowed to cool down to room temperature, cooled to 0° C. and extracted with ethyl acetate to remove non-polar impurities.

The reaction mixture was concentrated under reduced pressure to around 25% of the original volume. Sodium carbonate was then added to the reaction mixture until the pH of the mixture attained 10-11. The reaction mixture was then extracted with toluene at 80° C. The extract was concentrated under reduced pressure to furnish the amine V as a white solid; yield: 4 g, 62.5%.

### Example 3

### [3-Phenyl-4-(phenylcarbamoyl)-2-(4-fluorophenyl)-5-(1-methylethyl)-pyrrole-1-yl]methyl(diphenyl)phosphine oxide I

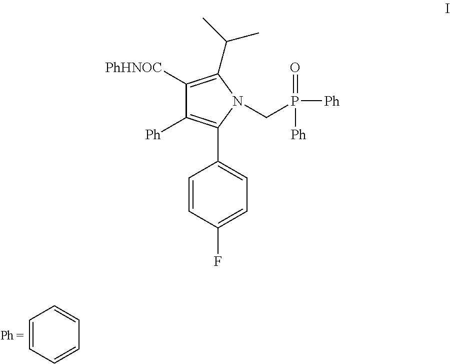

Toluene (45 ml) was added to a mixture of the amine V (3.7 g, 16.01 mmole), the atorvastatin 1,4-diketo compound VI (3.33 g, 7.98 mmole) and pivalic acid (0.24 g, 2.35 mmole) at room temperature. The reaction mixture was heated to reflux temperature. The water generated in the reaction was removed by using a Dean-Stark apparatus.

After 48 h at reflux, the reaction mixture was allowed to cool down to room temperature. The precipitated solid was filtered and washed with cold toluene to furnish the pyrrole derivative I as a white solid; yield: 2.6 g, 53.3%.

### Example 4

### Diethyl phthalimidomethylphosphonate VII

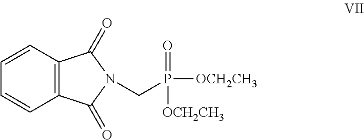

Triethyl phosphite (32 g, 192.59 mmol) was added to N-(bromomethyl)phthalimide III (46 g, 191.62 mmole) at room temperature and the reaction mixture was heated to 120° C. when vigorous reaction initiated by evolution of ethyl bromide gas.

After 1 h at 120° C., the reaction mixture was allowed to cool down to room temperature and diluted with chloroform (250 ml). The mixture was washed with water (3×100 ml), brine (3×100 ml), dried over sodium sulfate and concentrated to furnish the phosphonate compound VII as a thick oil which is solidified on standing under vacuum; yield: 40 g; 70%.

The crude material was carried over to the following step without further purification.

### Example 5

### Diethyl aminomethylphosphonate VIII

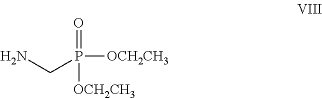

Hydrazine hydrate (4.58 g, 91.49 mmole) was added to a solution of the phosphonate VII (27 g, 90.90 mmole) in ethanol (57.8 ml) at room temperature.

The reaction mixture was stirred at this temperature until the reaction was complete as indicated from the TLC of the mixture. The reaction mixture was filtered and the filtrate was concentrated under reduced pressure to give the amine VIII as an oil; yield: 15 g, 71%.

### Example 6

### Diethyl [3-Phenyl-4-(phenylcarbamoyl)-2-(4-fluorophenyl)-5-(1-methylethy)-pyrrole-1-yl]methylphosphonate II

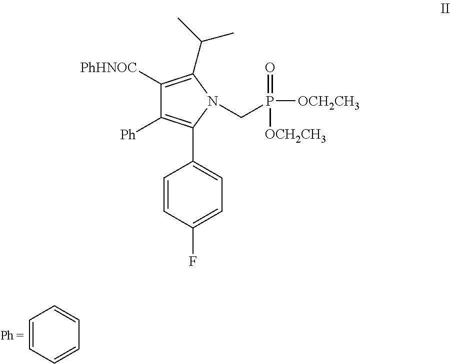

Pivalic acid (5.4 g, 52.87 mmole) was added to a solution of the amine VIII (10 g, 59.86 mmole) in tetrahydrofuran (20 ml) at room temperature (Scheme: 9). The diketone compound VI (11.2 g, 26.85 mmole) in tetrahydrofuran (20 ml) was then added to the mixture followed by addition of toluene (30 ml) and n-heptane (30 ml). The reaction mixture was heated to reflux with simultaneous removal of water generated during the reaction using a Dean-Stark apparatus. After completion of the reaction as indicated from the TLC, the reaction mixture was allowed to cool down to room temperature, concentrated under reduced pressure to a thick syrup and diluted with ethyl acetate. The organic layer was washed with water (3×10 ml), dilute hydrochloric acid (5×10 ml), water (3×10 ml), saturated sodium bicarbonate solution (3×10 ml), water (3×10 ml), brine (3×10 ml), dried over sodium sulfate and concentrated under reduced pressure to furnish the pyrrole derivate II; yield 23 g, 70%.

### Example 7

### Conversion of [3-Phenyl-4-(phenylcarbamoyl)-2-(4-fluorophenyl)-5-(1-methylethyl)-pyrrole-1-yl]methyl(diphenyl)phosphine oxide I, pyrrole derivative I into furanose moiety XI

Under appropriate Wittig reaction conditions the pyrrole I may react with the known aldehyde IX moiety to furnish the intermediate hydroxyl compound X, which on treatment with a suitable base, may be converted into the double bond containing furanose moiety XI (Scheme: 4).

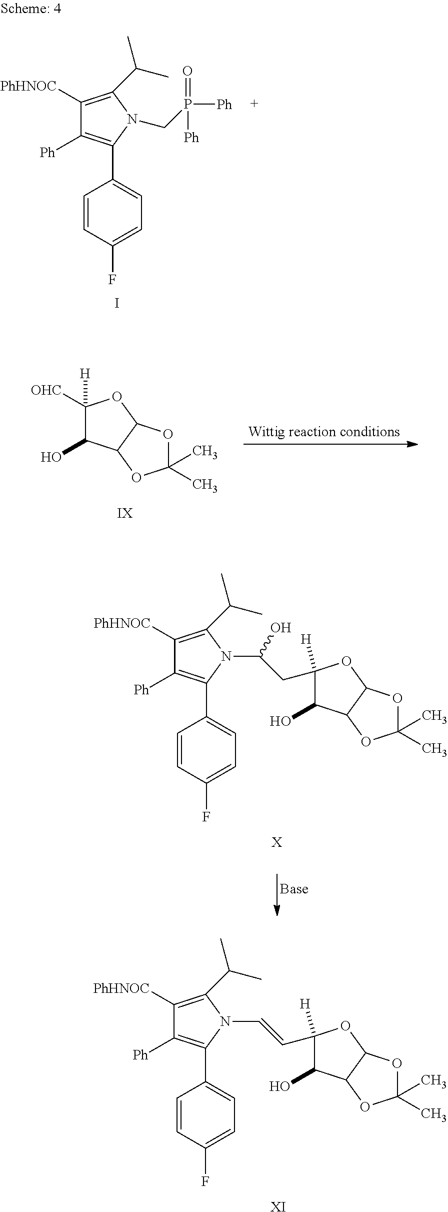

### Example 8

### Conversion of Diethyl [3-Phenyl-4-(phenylcarbamoyl)-2-(4-fluorophenyl)-5-(1-methylethyl)-pyrrole-1-yl]methylphosphonate II into the furanose moiety XI

The Diethyl [3-Phenyl-4-(phenylcarbamoyl)-2-(4-fluorophenyl)-5-(1-methylethyl)-pyrrole-1-yl]methylphosphonate II under Wittig conditions may be converted into furanose moiety XI in a single step without the intermediacy of the hydroxyl compound X, which was the case in the previous example.

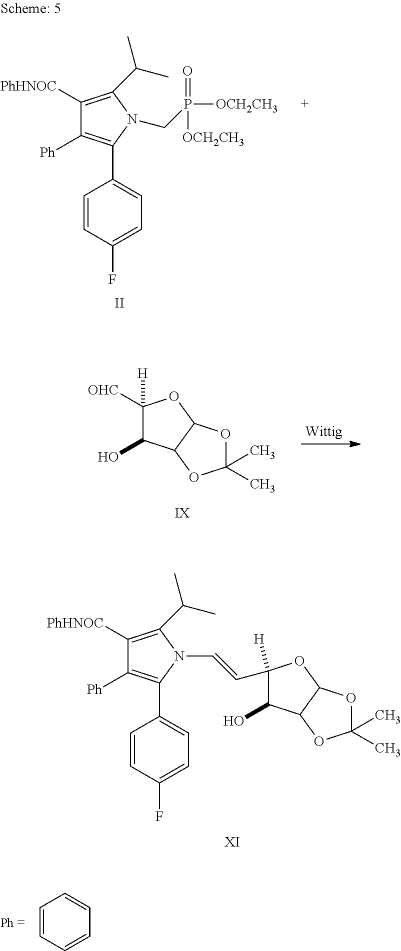

### Example 9

### The Conversion of Furanose Moiety XII into Atorvastatin

The reduction of the furanose derivatives XI should saturate its double bond to give its corresponding saturated furanose moiety XII. The compound XII serves as an important precursor for the synthesis of atorvastatin. The furanose moiety XII may be converted into atorvastatin by any known method e.g. as exemplified by PCT/GB2008/002008.

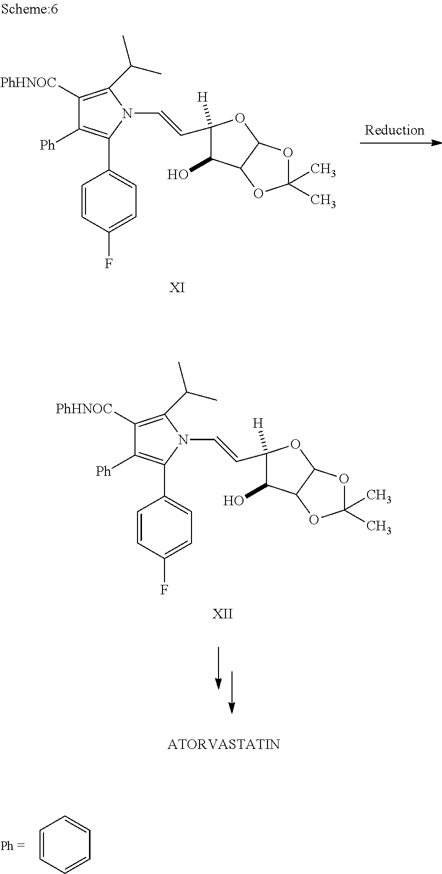

1. An isolated compound of formula I

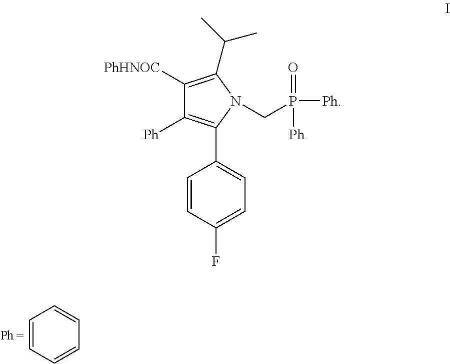

2. A process for preparing the compound of claim 1 comprising:
a. reacting ethyl diphenylphosphhimite in toluene with N-(bromomethyl)phthalimide 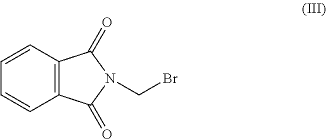
at room temperature to produce diphenyl(phtalimidomethyl) phosphine oxide 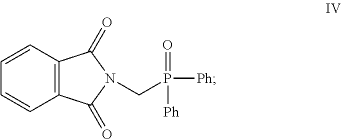
b. converting IV to amine 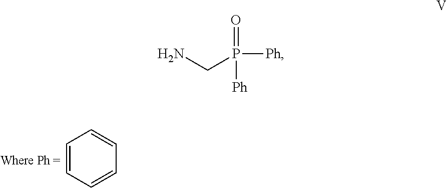
by reacting diphenyl(phtalimidomethyl) phosphine oxide IV with aqueous hydrobromic acid; and
c. condensing aminomethyl(diphenyl)phosphine oxide V with atorvastatin 1,4-diketo compound (2-[1-Phenyl-2-(4-fluorophenyl)-2-oxo-ethyl]-4-methyl-N-methyl-N-phenyl-3-oxo-pentamide) to form compound of formula I.

3. The process as claimed in claim 2 wherein the reaction of ethyl diphenylphosphinite in toluene and N-bromomethylphthalimide is carried out at 90° C. for 48 hours to produce diphenyl(phtalimidomethyl) phosphine oxide IV.

4. The process as claimed in claim 3 wherein diphenyl(phtalimidomethyl) phosphine oxide IV is reacted with aqueous hydrobromic acid while the the mixture is heated up to 100° C. for 15 hours; and
concentrating and adjusting the pH of the reaction mixture to a pH within 10-11 to extract out the amine V.

5. The process as claimed in any of claims 1 to 4 wherein the amine V is converted into the compound of formula I by reacting it with the atorvastatin 1,4-diketo compound (2-[1-Phenyl-2-(4-fluorophenyl)-2-oxo-ethyl]-4-methyl-N-methyl-N-phenyl-3-oxo-pentamide), in toluene catalysed by a suitable acid such as pivalic acid.

6. An isolated compound of formula II

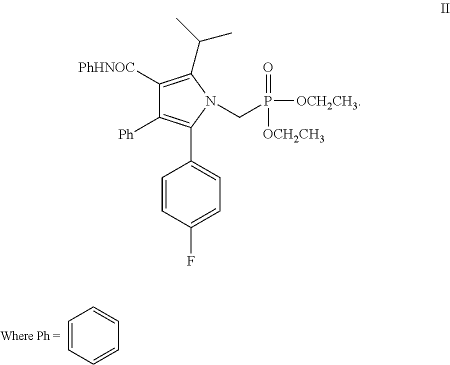

7. A process of obtaining an isolated compound of claim 6 comprising:
a. reacting triethyl phosphite with N-(bromomethy)lphthalimide to form phosphonate 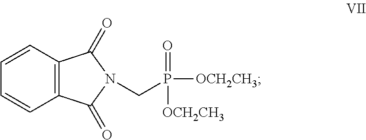
b. reacting the phosphonate VII obtained from (a) with hydrazine hydrate in ethanol to form the amine 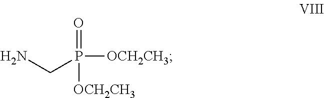
and
c. reacting the amine of (b) with atorvastatin 1,4-diketone (2-[1-Phenyl-2-(4-fluorophenyl)-2-oxo-ethyl]-4-methyl-N-methyl-N-phenyl-3-oxo-pentamide) in toluene catalysed by pivalic acid to form the compound of formula II.

8. The process as claimed in claim 7, wherein the triethyl phosphite is reacted with said N-(bromomethyl)phthalimide at a temperature of about 120° C. for about 1 hour, allowing the reaction mixture to cool down to room temperature, diluting the reaction mixture with chloroform, washing with water and brine and drying the reaction mixture over sodium sulfate to form the phosphonate VII.

9. The process as claimed in claim 8, wherein the phosphonate VII is reacted with hydrazine hydrate in ethanol for 48 hours and concentrated under reduced pressure to form the amine VIII.

10. The process of any of claims 7 to 9, wherein the amine VIII is reacted with the atorvastatin 1,4- diketone (2-[1-Phenyl-2-(4-fluorophenyl)-2-oxo-ethyl]-4-methyl-N-methyl-N-phenyl-3-oxo-pentamide) in a solvent system comprising toluene and n-heptane, concentrating the reaction mixture under reduced pressure and further diluting the reaction mixture with ethyl acetate to produce a diluted mixture.

11. The process as claimed in claim 10 wherein the organic layer of the diluted mixture is washed with water, hydrochloric acid and sodium bicarbonate solution, and the resultant solution is dried under low pressure over sodium sulfate to obtain compound pyrrole derivative II.

12. A method of synthesizing atorvastatin, said method comprising the step of using a compound selected from the group consisting of 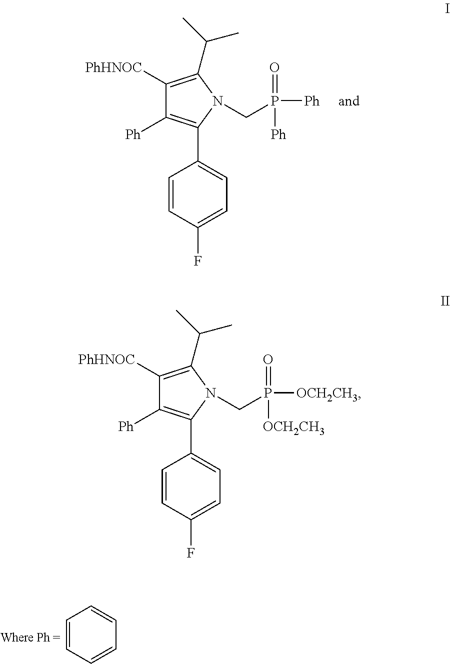
as an intermediate to produce furanose derivative XI: 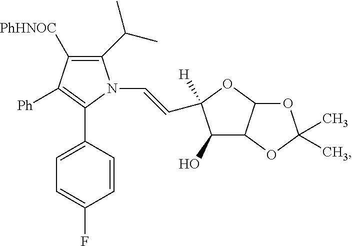
which is then converted to atorvastatin.
